(19) United States
(12) Patent Application Publication
(10) Pub. No.: US 2026/0124265 A1
(43) Pub. Date: May 7, 2026

(54) Formulation For Treatment of Middle Ear Infections Without Antibiotics

(71) Applicant: Tara Chand Singhal, Torrance, CA (US)

(72) Inventor: Tara Chand Singhal, Torrance, CA (US)

(21) Appl. No.: 19/438,856

(22) Filed: Jan. 2, 2026

Related U.S. Application Data
(63) Continuation of application No. 15/434,362, filed on Feb. 16, 2017, now abandoned.
Application No. 15/434,362 is a continuation-in-part of application No. 13/850,167, filed on Mar. 25, 2013, now Pat. No. 10,300,258.
(60) Provisional application No. 61/719,961, filed on Oct. 30, 2012.
(60) Provisional application No. 62/307,442, filed on Mar. 12, 2016.

(51) Int. Cl. A61K 36/31; A61K 9/00; A61K 36/53; A61M 31/00; B65D 35/28; B65D 39/00; B65D 75/58; B65D 85/07

(52) U.S. Cl. CPC A61K 36/31; A61K 9/0046; A61K 36/53; A61M 31/00; B65D 35/28; B65D 75/5805; B65D 85/07; A61M 2205/36; A61M 2210/0668; B65D 39/00; B65D 2581/3471; B65D 2583/00

(57) ABSTRACT

A formulation for treating middle ear infections is a blend of mustard oil and lavender essential oil, for application in treating middle ear infections. A proportion in the formulation of lavender oil to the mustard oil, by volume is from, 5% to 45%.

### CROSS REFERENCE

This application is a continuation of Ser. No. 15/434,362, filed on Feb. 15, 2017 of Tara Chand Singhal, for A Formulation For Treatment of Middle Ear Infections Without Antibiotics, which application is a continuation-in-part (CIP) of and claims priority from application Ser. No. 13/850,167, filed on Mar. 25, 2013 of Tara Chand Singhal, for Apparatus and Method for Natural Cure of Middle Ear Infections Without Antibiotics, now U.S. Pat. No. 10,300,258, issued on May 28, 2019. The contents of application Ser. No. 13/850,167 are incorporated herein by reference.

Application Ser. No. 15/434,362 claims priority from Provisional application Ser. No. 62/307,442, filed on Mar. 12, 2016 of Tara Chand Singhal, for A Compound for treatment of Middle Ear Infections. The contents of application Ser. No. 62/307,442 are incorporated herein by reference.

Application Ser. No. 13/850,167 claims priority from Provisional application Ser. No. 61/719,961, filed on Oct. 30, 2012 of Tara Chand Singhal, for Apparatus and Method for Natural Cure of Middle Ear Infections Without Antibiotics. The contents of application Ser. No. 61/719,961 are incorporated herein by reference.

### FIELD OF THE INVENTION

A formulation for the treatment of middle ear infections that uses a blend of mustard oil and lavender essential oil is described.

### BACKGROUND

Next to the common cold, ear infections are the most commonly diagnosed childhood illness in the United States. More than 3 out of 4 kids have had at least one ear infection by the time they reach 3 years of age. Adults are not immune from ear infections even though they get it much less frequently.

To understand how ear infections develop, let's review how the ear works. Every time one hears a sound, the various structures of the ear have to work together to make sure the information gets to the brain.

The ear is responsible for hearing and balance and is made up of three parts—the outer ear, middle ear, and inner ear. Hearing begins when sound waves that travel through the air reach the outer ear, or pinna, which is the part of the ear that's visible. The sound waves then travel from the pinna through the ear canal to the middle ear, which includes the eardrum (a thin layer of tissue) and three tiny bones called ossicles. When the eardrum vibrates, the ossicles amplify these vibrations and carry them to the inner ear.

The inner ear translates the vibrations into electric signals and sends them to the auditory nerve, which connects to the brain. When these nerve impulses reach the brain, they're interpreted as sound. To function properly, the middle ear must be at the same pressure as the outside world. This is taken care of by the Eustachian tube, a small passage that connects the middle ear to the back of the throat behind the nose.

By letting air reach the middle ear, the Eustachian tube equalizes the air pressure in the middle ear to the outside air pressure. (When your ears “pop” while yawning or swallowing, the Eustachian tubes are adjusting the air pressure in your middle ears.) The Eustachian tube also allows for drainage of mucus from the middle ear into the throat. Sometimes, the Eustachian tube may malfunction. For example, when someone has a cold or an allergy affecting the nasal passages, the Eustachian tube may become blocked by congestion in its lining or by mucus within the tube. This blockage will allow fluid to build up within the normally air-filled middle ear.

Bacteria or viruses that have entered the middle ear through the Eustachian tube also can get trapped in this way. These germs can breed in the trapped fluid, eventually leading to an ear infection.

An ear infection (acute otitis media) is most often a bacterial or viral infection that affects the middle ear, the air-filled space behind the eardrum that contains the tiny vibrating bones of the ear. Children are more likely than adults to get ear infections.

Ear infections are often painful because of inflammation and buildup of fluids in the middle ear. The onset of signs and symptoms of ear infection is usually rapid. Signs and symptoms of an ear infection can indicate a number of different conditions.

An ear infection is caused by a bacterium or virus in the middle ear. This infection often results from another illness—cold, flu or allergy—that causes congestion and swelling of the nasal passages, throat and Eustachian tubes.
The Eustachian tubes are a pair of narrow tubes than run from each middle ear to high in the back of the throat, behind the nasal passages. Swelling, inflammation and mucus in the Eustachian tubes from an upper respiratory infection or allergy can block them, causing the accumulation of fluids in the middle ear. A bacterial or viral infection of this fluid is usually what produces the symptoms of an ear infection.

Ear infections are more common in children, in part, because their Eustachian tubes are narrower and more horizontal—factors that make them more difficult to drain and more likely to get clogged.

Middle ear infection is common among people of all ages. The infection may arise due to many causes such as infection of throat, and whenever the bacterium makes its way inside the ear canal.

The middle ear infection is quite painful and requires a visit to a doctor in an urgent care situation. The middle part of the ear is unreachable by the patient and cannot be treated by external use of ointments.

The doctor after examination of the ear canal and seeing redness associated with infection usually prescribes an antibiotic. The antibiotic treatment is usually taken for a set number of days, for example seven days, to get rid of the infection.

Based on medical science history, bacteria is known to develop resistance to antibiotics and hence for this reason medical profession rather not over prescribe antibiotics for conditions that can be cured otherwise. Unfortunately, western medical science has no alternative cure for middle ear infection.

It is the objective of the embodiments herein to have a cure for middle ear infection that does not require use of antibiotic. It is further the objective to treat the infection without a visit to the doctor and much sooner than seven days. It is yet another objective to cure the middle ear infection at much lower cost than the combination of doctor fees and cost of antibiotics.

Next to the common cold, ear infections are the most commonly diagnosed childhood illness in the United States. More than 3 out of 4 kids have had at least one ear infection by the time they reach 3 years of age. Adults are not immune from ear infections even though they get it much less frequently.

To function properly, the middle ear must be at the same pressure as the outside world. This is taken care of by the Eustachian tube, a small passage that connects the middle ear to the back of the throat behind the nose.

For example, when someone has a cold or an allergy affecting the nasal passages, the Eustachian tube may become blocked by congestion in its lining or by mucus within the tube. This blockage will allow fluid to build up within the normally air-filled middle ear.

Bacteria or viruses that have entered the middle ear through the Eustachian tube also can get trapped in this way. These germs can breed in the trapped fluid, eventually leading to an ear infection.

An ear infection (acute otitis media) is most often a bacterial or viral infection that affects the middle ear, the air-filled space behind the eardrum that contains the tiny vibrating bones of the ear. Children are more likely than adults to get ear infections.

Ear infections are often painful because of inflammation and buildup of fluids in the middle ear. The onset of signs and symptoms of ear infection is usually rapid. Signs and symptoms of an ear infection can indicate a number of different conditions.

An ear infection is caused by a bacterium or virus in the middle ear. This infection often results from another illness—cold, flu or allergy—that causes congestion and swelling of the nasal passages, throat and Eustachian tubes.

Ear infections are more common in children, in part, because their Eustachian tubes are narrower and more horizontal—factors that make them more difficult to drain and more likely to get clogged.

The middle ear infection is quite painful and requires a visit to a doctor in an urgent care situation. The middle part of the ear is unreachable by the patient and cannot be treated by external use of ointments.

The doctor after examination of the ear canal and seeing redness associated with infection usually prescribes an antibiotic. The antibiotic treatment is usually taken for a set number of days, for example seven days, to get rid of the infection.

Based on medical science history, bacteria is known to develop resistance to antibiotics and hence for this reason medical profession rather not over prescribe antibiotics for conditions that can be cured otherwise. Unfortunately, western medical science has no alternative cure for middle ear infection.

It is the objective of the embodiments herein to have a treatment and a cure for middle ear infection that does not require use of antibiotic. It is further the objective to treat the infection without a visit to the doctor and much sooner than seven days. It is yet another objective to cure the middle ear infection at much lower cost than the combination of doctor fees and cost of antibiotics.

### SUMMARY

Middle ear infections can be quickly ameliorated with use of mustard essential oil. The embodiments describe here are an apparatus and a method for a single application for a natural cure for middle ear infection using mustard essential oil.

Essential oil is extracted by steam distillation of mustard seeds soaked in water. Alternatively for this application cold-pressed oil of mustard seeds may also be used.

Mustard essential oil has bactericidal or anti bacterial properties. Internally it fights bacterial infections in the colon, digestive system, excretory system, urinary tract etc. Externally applied, it can treat bacterial infections on skin. For tutorial purposes, the following extract from Internet describes the various properties of mustard oil or essential oil is copied here.

Black and white mustards are used for culinary and medicinal purposes. The leaves, flowers, seeds, and oils of the black mustard are used, while only the seeds of the white mustard are useful. Black mustard powder is an important herbal remedy because it draws blood to the surface of the skin quickly, which means that it is rubefacient, and is warming. Mustard essential oil is used as an ingredient in liniments, stomach stimulants, and emetics.

Mustard is rich in many vitamins and nutrients that are beneficial to everyone. Mustard is rich in calcium and iron and helps to restore bacterial balance in the intestines. The mustard's greens are rich in vitamin A, iron, and zinc, and are best when eaten raw or steamed. White mustard has been used throughout the world to relieve pain, and as a diuretic and an antibiotic Mustard flour is an antiseptic and can also be used as a deodorizer. The mustard's oil can be used for pain relief of arthritic conditions and chilblains. Mustard is also an excellent expectorant and a very powerful natural emetic.

Some of the most valuable properties of mustard lie in its warming and rubefacient properties. Black mustard and white mustard can be used to draw infection or congestion away from its source for nasal congestion, or for relief of an abscess. The rubefacient qualities help respiratory and circulatory disorders, including some heart problems.

As described in the embodiments herein, the middle ear infections can be quickly ameliorated with use of mustard essential oil. The embodiments describe here an apparatus for natural cure for middle ear infection. The apparatus has a single use sachet. The sachet has a pocket and the pocket holds a limited quantity of mustard essential oil. The sachet has a neck and a dropper and sachet can be squeezed to flow drops for pouring into an ear canal.

The mustard essential oil preferably is heated to about 115 degrees Fahrenheit. Therefore, the sachet is put in a microwave oven just before use for only substantially 30 seconds to heat the oil and up to five drops of heated oil is squeezed into an ear canal of a person with severe ear itching indicative of a middle ear infection.

The head is positioned on a side to have one ear facing up before dropping the oil in the ear canal and the head is stayed in position for substantially five minutes to have the oil flow deep in to the middle ear. The single use sachet is in a packaging with two cotton ear plugs.

The ear is plugged with the cotton plug and head is turned on the other side for dropping the oil into the second ear. This method of treatment of middle ear infection usually provides an instant relief from the itching and usually cures the infection in a single treatment as above. These and other aspects of the embodiments are described in detail here with the help of the accompanying figures.

The embodiments herein describe a blend of mustard oil and lavender essential oil for treating middle ear infections.

Mustard oil has bactericidal or anti bacterial properties. Externally applied, it can treat bacterial infections on skin. Some of the most valuable properties of mustard lie in its warming and rubefacient properties.

Lavender essential oil has antiseptic and soothing properties and is used for common skin applications such as for the bruises, burns, dermatitis, eczema, hives, inflammations, insect bites and stings, and sunburns.

The embodiments herein described are for a formulation for treatment and cure for middle ear infections. The formulation uses and has a blend of mustard oil and lavender essential oil. The proportion of lavender oil and the mustard oil in the formulation may vary, by volume, from 5% to 45% of the lavender oil in the mustard oil.

Before use, the formulation preferably is warmed to about 110 to 115 degrees Fahrenheit for a therapeutic effect. There may be many methods for warming the oil. These methods may include the container with the formulation is put in a microwave oven just before use for only substantially 30 seconds or less to warm the oil. Alternatively the formulation may be warmed up by placing the container in hot water for some time.

Up to five drops of warm formulation oil is squeezed into an ear canal of a person with severe ear itching indicative of a middle ear infection. The head is positioned on a side to have one ear facing up before dropping the oil in the ear canal and the head is stayed in position for some time for the formulation oil to flow through to the middle ear. These and other aspects of the embodiments are described in detail here with the help of the accompanying figures.

### BRIEF DESCRIPTION OF THE DRAWINGS

Some of the novel features of this invention will be best understood from the accompanying drawings, taken in conjunction with the accompanying description, in which similar reference characters refer to similar parts, and in which:

FIG. 1A is a block diagram that illustrates features of the present invention of a sachet for cure of middle ear infection;

FIG. 1B is a block diagram that illustrates features of the present invention of using a sachet for cure of middle ear infection;

FIG. 2A is a block diagram that illustrates features of the present invention of packaging of a sachet for cure of middle ear infection;

FIG. 2B is a block diagram that illustrates features of the present invention of dropper of a sachet for cure of middle ear infection;

FIG. 3A is a block diagram that illustrates features of labels on packaging for sachet for middle ear infection cure;

FIG. 3B is a block diagram that illustrates features of instructions for use of sachet for middle ear infection cure;

FIG. 4A is a block diagram that illustrates features of the present invention of a method of heating the oil in the sachet for middle ear infection cure;

FIG. 4B is a block diagram that illustrates features of the present invention of another method of heating the oil in the sachet for middle ear infection cure;

FIG. 5 is a method diagram that illustrates features of the present invention of a cure for middle ear infection;

FIG. 6 is a block diagram that illustrates features of the present invention of a container with a formulation for cure of middle ear infection;

FIG. 7 is a block diagram that illustrates features of the present invention of using a formulation for cure of middle ear infection;

FIG. 8 is a method diagram that illustrates features of the present invention of a cure for middle ear infection.

### DESCRIPTION

The embodiments described herein are directed to a natural cure for middle ear infection. The natural cure uses mustard essential oil and avoids use of antibiotics.

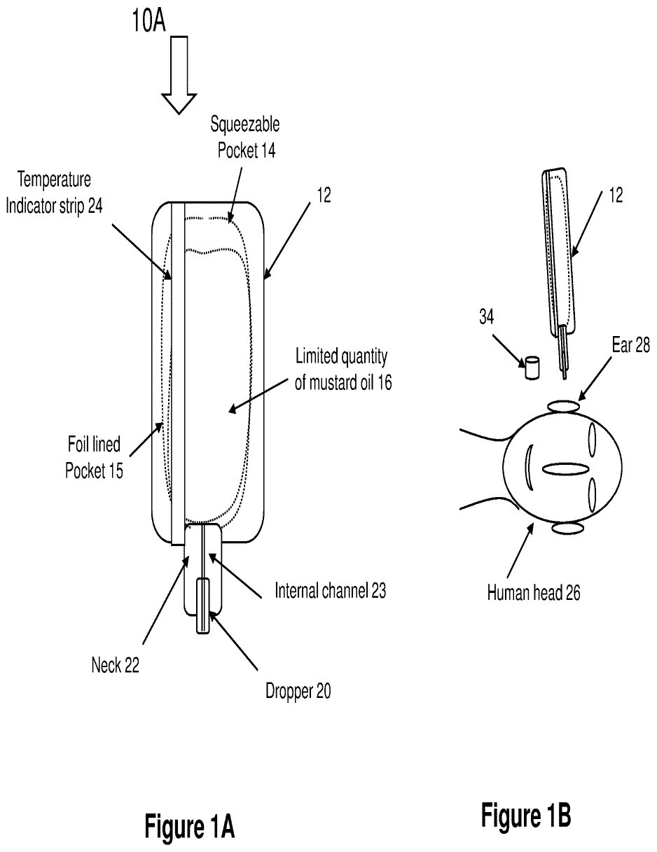

With reference to FIG. 1A, an apparatus 10A for natural cure for middle ear infection is single use sachet 12. The sachet 12 holds limited quantity of mustard essential oil 16 inside a foil lined pocket 15. The foil lined pocket 15 is a squeezable pocket 14 to be able to squeeze drops of oil via the dropper 20 attached to neck 22.

FIG. 1B illustrates use of sachet 12 with a human head 26 positioned on the side exposing one of the human ear 28. An ear plug 34 is used after the oil had been put in the ear 28.

As illustrated in FIG. 1A, the sachet 12 has a pouch 14 and the pouch is sized to and holds a limited quantity of mustard essential oil 16. The quantity of mustard essential oil is limited to notionally ten drops and could be little more to account for that not all may be useable. The sachet 12 has a neck 22 and a dropper 20. The sachet 12 can be squeezed to flow drops of mustard essential oil for dropping into an ear canal via the dropper 20. The neck 22 provides a length that is suitable to be able to easily access the outer ear canal.

Similar sachets are used in prior art for application such as for dispensing beauty products, creams and medicinal ointments etc to the palm of the hand and/or fingers. Hence the technology of making such sachets is considered prior art.

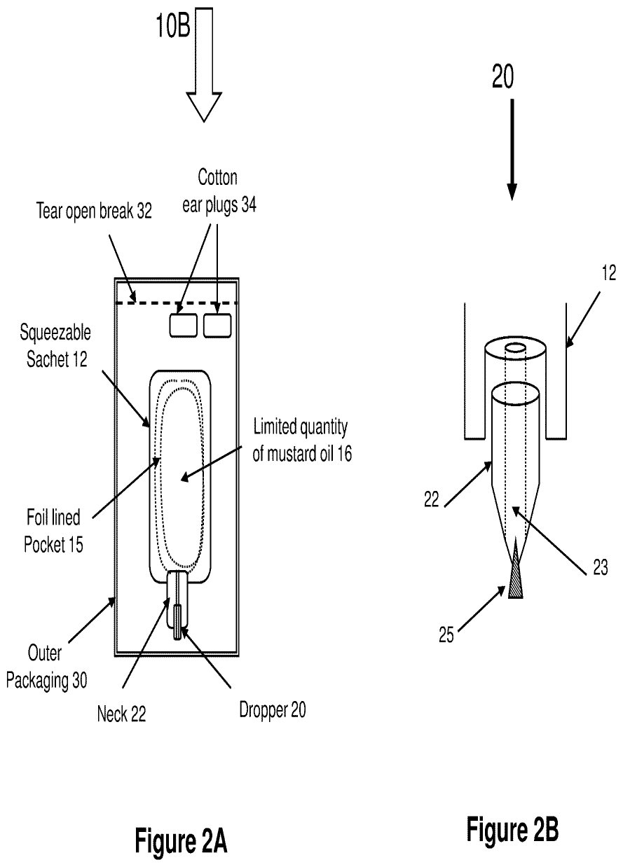

As illustrated in FIG. 2A, in embodiment 10B, the sachet 12 is packaged inside a package 30 and the package 30 has a tear open side 32 to be able to tear open package 30.

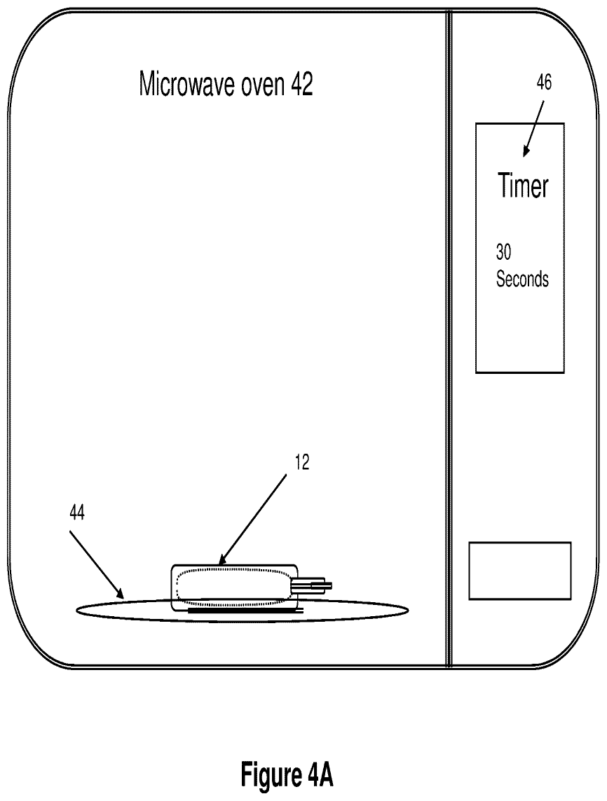

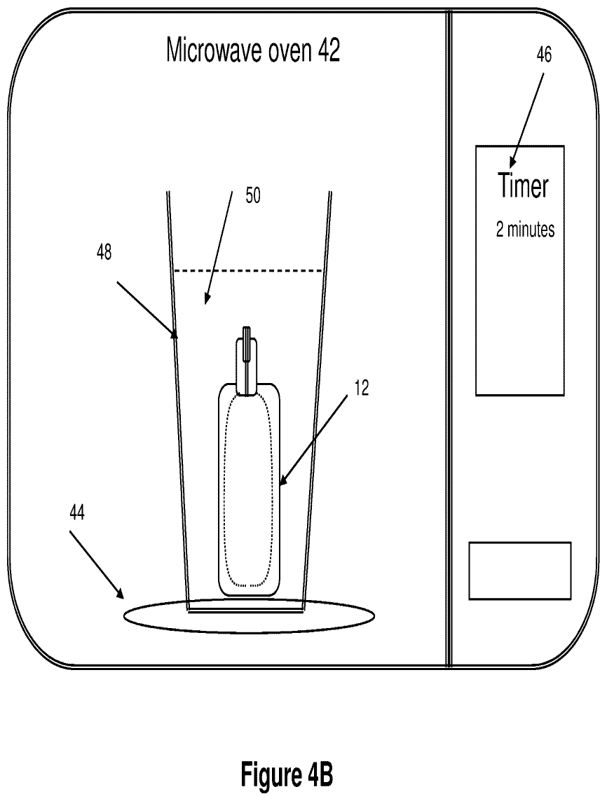

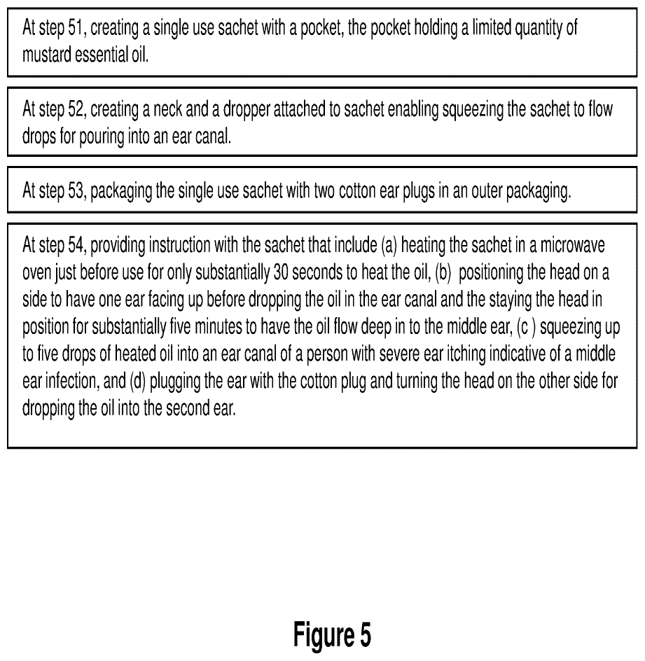

As illustrated later, with the help of FIGS. 4A and 4B, the sachet 12 and thus the oil 16 inside the sachet is heated to a comfortable therapeutic temperature of around 110 to 115 degrees.

As illustrated in FIG. 1A, optionally the sachet 12 has attached a liquid crystal thermometer strip 24 which provides for an advisory that the oil is at a suitable temperature before the sachet should be used. The science of liquid crystal thermometer is prior art. A liquid crystal thermometer or plastic strip thermometer is a type of thermometer that contains heat-sensitive liquid crystals in a plastic strip that change color to indicate different temperatures.

Liquid crystals possess the mechanical properties of a liquid, but have the optical properties of a single crystal. Temperature changes can affect the color of a liquid crystal, which makes them useful for temperature measurement. The resolution of liquid crystal sensors is in the 0.1° C. range. Disposable liquid crystal thermometers have been developed for home and medical use. For example if the thermometer is black and it is put onto someone's forehead it will change color depending on the temperature of the person.

Liquid crystal thermometers portray temperatures as colors and can be used to follow temperature changes caused by heat flow. In medical applications, liquid crystal thermometers may be used to read body temperature by placing against the forehead.

In the application of the embodiments herein, the thermometer strip 24 is used to indicate to a user, as a precautionary measure, that the oil 16 is not hotter than in the range of 110 to 115 degrees at the time of dropping the oil in the ear canal. This may be accomplished in other ways as well as such as by hand feel. The oil when heated may be above 115 degrees as that temperature would drop before use.

As shown in FIG. 2A, the single use sachet 12 has in the packaging 30, two cotton ear plugs 34. These ear plugs 34 are intended to and used to plug the ear canal from the oil that may come out from the ear canal.

Alternatively, the sachet 12 may be in the form of a bottle (not shown) and different such embodiments are not ruled out. However, the sachet 12 is preferred, as given small quantity of mustard essential oil, a sachet given its size may be more convenient to handle and use. The sachet 12 may be one inch wide and two inches high. It is believed that such a size of sachet 12 would be convenient to hold and use for its intended purpose.

The ear plugs 34 are preferably cotton to absorb the oil that may leak out of the ear canal. However, other absorbent materials are not ruled out.

With reference to FIG. 2B, a close up of the construction of the dropper 20 is illustrated. The sachet 12 is integrally attached to a neck 22. The neck 22 has a channel 23 for squeezing the oil from the pocket 14 via the channel 23 to the ear canal when the pocket 14 with oil 16 is squeezed. A stopper 25 is attached to the end of the channel 23 and is removed or broken away before use.

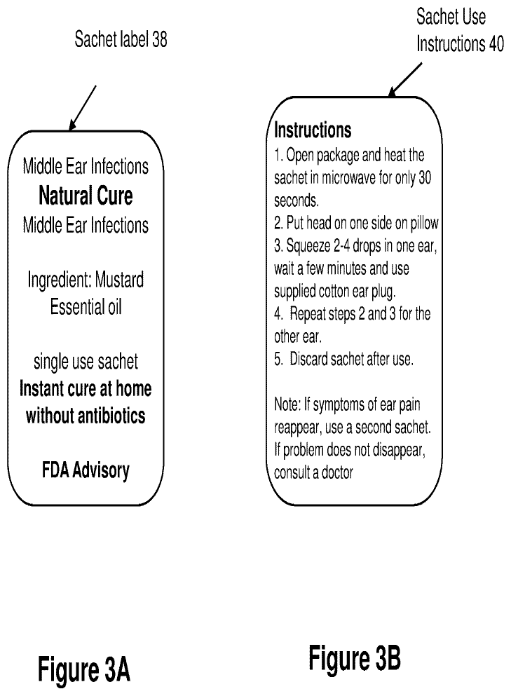

With reference to FIG. 3A, content of a label 38 on the sachet 12 is illustrated. The label 38 provides for a label informing a user about the contents and purpose of the package 30 as well as an FDA advisory.

With reference to FIG. 3B, content of an instruction 40 for the use of the sachet 12 is illustrated. The use instruction 40 provides:
1. Open package and heat the sachet in microwave for only 30 seconds.
2. Put head on one side on pillow
3. Squeeze 2-4 drops in one ear, wait a few minutes and use supplied cotton ear plug.
4. Repeat steps 2 and 3 for the other ear.
5. Discard sachet after use.
Note: If symptoms of ear pain reappear, use a second sachet.
If problem does not disappear, consult a doctor

The sachet 12 is made of a material that can be put inside a microwave oven and heat the oil in the pouch. As illustrated with the help of FIG. 4A, if the sachet 12 is made of plastic, the sachet can be directly placed on the tray 44 of a microwave oven 42.

Alternatively as illustrated in FIG. 4B, if the sachet 12 is made of metal foil, the sachet 12 can also be heated in a glass 48 with about 8 OZ of water 50 inside the microwave oven 42.

Based on these instructions, the sachet is put in a microwave oven just before use for only notionally 30 seconds to heat the oil in the sachet up to in the range of 110 to 115 degrees. It is believed that such a limited use of microwave oven would heat the oil for this purpose. Notionally ten to twenty degrees of temperature over the body temperature of 98.4 degrees provides for warm oil and not hot oil. This degree of warmness of oil provides for a therapeutic effect when the warn oil drops enter the ear canal.

Temperature of the sachet and the oil may be higher when first heated in the microwave oven than this notionally 110 to 115 degrees. The temperature of the oil would drop when the sachet is removed from the microwave oven and before it is used.

The head of a person with middle ear infection is positioned on a side to have one ear facing up before dropping the oil in the ear canal and the head is stayed in position for substantially a few minutes to have the oil flow in to the middle ear.

Up to five drops of heated oil is squeezed into an ear canal of a person with severe ear itching indicative of a middle ear infection. Fewer drops, such as one to three may be used for children. The ear is plugged with the cotton plug 18 and head is turned on the other side for dropping the oil into the other ear.

An apparatus for natural cure for middle ear infection has a single use sachet. The sachet has a pouch and the pouch holds a limited quantity of mustard essential oil. The sachet has a neck and a dropper and sachet can be squeezed to flow drops of the oil for dropping into an ear canal. The single use sachet is packaged in a packaging with two absorbent ear plugs.

The oil is warmed up to in the range of 110 to 115 degrees to provide a therapeutic effect as the oil is dropped in the ear canal. The head is positioned on a side to have one ear facing up before dropping the oil in the ear canal and the head is stayed in position for substantially a few minutes to have the oil flow in to the middle ear. The ear is plugged with the cotton plug and head is turned on the other side for dropping the oil into the second ear.

The sachet is put in a microwave oven just before use for only substantially 15 seconds to heat the oil to be warm. Up to five drops of heated oil from the sachet is squeezed into an ear canal of a person with severe ear itching indicative of a middle ear infection.

An apparatus for natural cure for middle ear infection has a single use sachet. The sachet has a pouch and the pouch holds a limited quantity of mustard essential oil. The sachet has a neck and a dropper and sachet can be squeezed to flow drops for dropping the oil into an ear canal. The oil, while in the sachet, is heated to a therapeutic temperature before being used.

The sachet has attached to it a liquid crystal thermometer strip for indicting the temperature of the oil. The thermometer is used to gauge the temperature of oil as being below 115 degrees before use in the ear canal when the sachet and the oil have been heated to a therapeutic temperature.

The sachet is put in a microwave oven just before use for only substantially 30 seconds to warm the oil and up to five drops of heated oil is squeezed into an ear canal of a person with severe ear itching indicative of a middle ear infection. The oil is warmed up to be in the range of 110 to 115 degrees to provide a therapeutic effect as the oil is dropped in the ear canal.

A method is illustrated with reference to FIG. 5, where not all the steps may be used or used in the order specified.
At step 51, creating a single use sachet with a pocket, the pocket holds a limited quantity of mustard essential oil.
At step 52, creating a neck and a dropper attached to sachet enabling squeezing the sachet to flow drops for pouring into an ear canal.
At step 53, packaging a single use sachet 12, in an outer packaging, along with two cotton ear plugs.
At step 54, providing instruction with the sachet that include (a) heating the sachet in a microwave oven just before use for only substantially 30 seconds to heat the oil, (b) positioning the head on a side to have one ear facing up before dropping the oil in the ear canal and the staying the head in position for substantially five minutes to have the oil flow deep in to the middle ear, (c) squeezing up to five drops of heated oil into an ear canal of a person with severe ear itching indicative of a middle ear infection, and (d) plugging the ear with the cotton plug and turning the head on the other side for dropping the oil into the second ear.

This method of treatment of middle ear infection, it is believed, usually provides an instant relief from the itching and usually cures the infection in a single treatment as above.

The embodiments described herein are directed to a treatment and a cure for middle ear infection. The embodiments described herein are for a formulation of mustard oil and lavender essential oil.

The formulation is a blend of mustard oil and lavender essential oil, for application in treating middle ear infections. It is believed a formulation of mustard oil and lavender essential oil provides an effective blend with both anti-bacterial properties of mustard oil and soothing properties of lavender oil.

Mustard oil is the main ingredient in the formulation. A proportion in the formulation of lavender oil to the mustard oil, by volume, is from, 5% to 45%.

The proportion of mustard oil and lavender oil in the formulation may be from one of (i) 55% mustard oil, 45% Lavender oil, (ii) 60% mustard oil, 40% lavender oil, (iii) 65% mustard oil, 35% lavender oil, (iv) 70% mustard oil, 30% lavender oil, (v) 75% mustard oil, 25% Lavender oil, (vi) 80% mustard oil, 20% lavender oil, (vii) 85% mustard oil, 15% lavender oil, (ix) 90% mustard oil, 10% lavender oil, (x) 95% mustard oil 5% lavender oil.

It is believed that based on the properties of mustard oil and lavender essential oil, a preferred proportion of mustard oil and lavender essential oil in the formulation may be either, 75% mustard oil, 25% Lavender oil, or 80% mustard oil, 20% lavender oil, or 85% mustard oil, 15% lavender oil.

The packaging for the formulation may be suitable size glass bottle used for essential oils. The formulation is packaged in a container that holds a limited quantity of the formulation, wherein the container has a neck and a dropper to flow drops of the oil for dropping into an ear canal.

The container is of material for warming the formulation before application, wherein the formulation is warmed up to in the range of 110 to 115 degrees to provide a therapeutic effect as the oil is dropped in the ear canal. The formulation may be warmed by putting the container in bowl of hot water or held under running hot water for a few minutes.

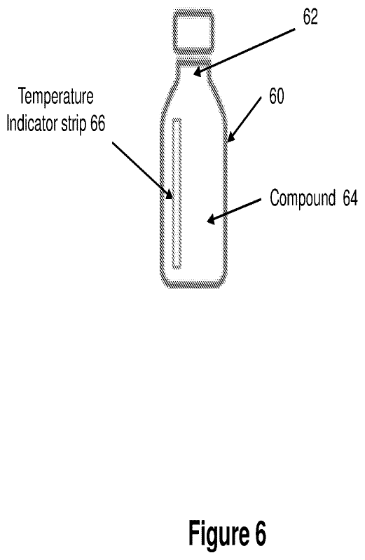

FIG. 6 illustrates a container 60 for the formulation 64. The container 60 may be glass bottle with a dropper 62 and may optionally have a temperature strip 66 that would indicate the temperature of the container 60 and thus the formulation 64 when the formulation 64 is warmed just before the application.

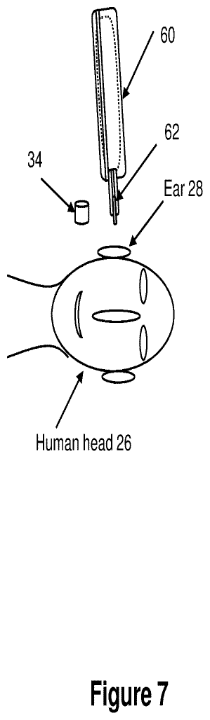

FIG. 7 illustrates use of the container 60 with the dropper 62 with a human head 26 positioned on the side exposing one of the human ear 28. An ear plug 34 is used after the formulation had been put in the ear 28.

The ear plugs 34 are preferably cotton to absorb the formulation that may leak out of the ear canal. However, other absorbent materials are not ruled out.

The head of a person with middle ear infection is positioned on a side to have one ear facing up before dropping the formulation in the ear canal and the head is stayed in position for substantially a few minutes to have the formulation flow in to the middle ear. The treatment is then repeated for the other ear

Up to five drops of heated formulation is squeezed into an ear canal of a person with severe ear itching indicative of a middle ear infection. Fewer drops, such as one to three may be used for children. The ear is plugged with the cotton plug 34 and head is turned on the other side for dropping the formulation into the other ear.

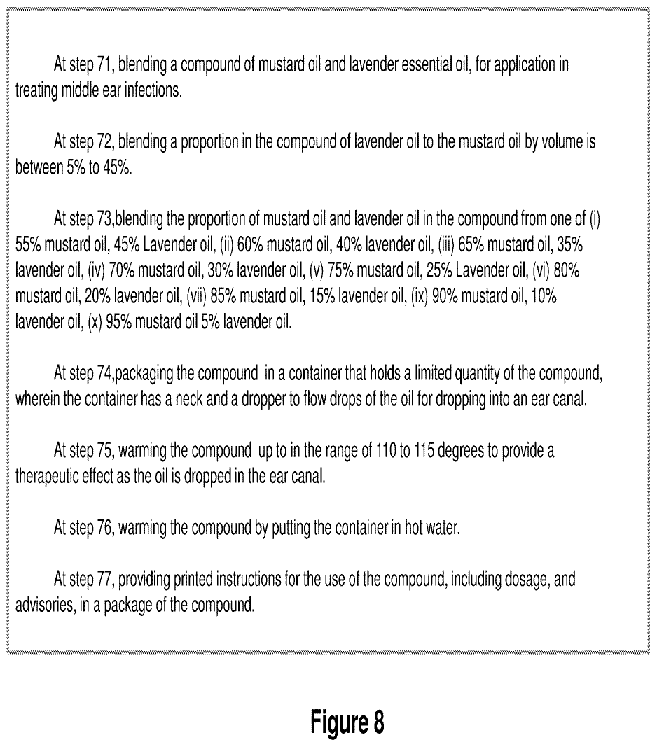

FIG. 8 illustrates a method for the formulation, where all the steps may not be used or used in the order specified.
At step 71, blending formulation of mustard oil and lavender essential oil, for application in treating middle ear infections.
At step 72, blending a proportion in the formulation of lavender oil to the mustard oil by volume is from 5% to 45%.
At step 73, blending the proportion of mustard oil and lavender oil in the formulation from one of (i) 55% mustard oil, 45% Lavender oil, (ii) 60% mustard oil, 40% lavender oil, (iii) 65% mustard oil, 35% lavender oil, (iv) 70% mustard oil, 30% lavender oil, (v) 75% mustard oil, 25% Lavender oil, (vi) 80% mustard oil, 20% lavender oil, (vii) 85% mustard oil, 15% lavender oil, (ix) 90% mustard oil, 10% lavender oil, (x) 95% mustard oil 5% lavender oil.
At step 74, packaging the formulation in a container that holds a limited quantity of the formulation, wherein the container has a neck and a dropper to flow drops of the oil for dropping into an ear canal.
At step 75, warming the formulation up to in the range of 110 to 115 degrees to provide a therapeutic effect as the formulation is dropped in the ear canal.
At step 76, warming the formulation by storing the container, for some time, in a bowl of hot water.
At step 77, providing printed instructions for the use of the formulation, including dosage, and advisories, in a package of the formulation.

While the particular invention, as illustrated herein and disclosed in detail is fully capable of obtaining the objective and providing the advantages herein before stated, it is to be understood that it is merely illustrative of the presently preferred embodiments of the invention and that no limitations are intended to the details of construction or design herein shown other than as described in the appended claims.

What is claimed is:

1. A formulation for treating middle ear infections, comprising:
the formulation is a blend of mustard oil and lavender essential oil, for application in treating middle ear infections.

2. The formulation, as in claim 1, comprising
a proportion in the formulation of lavender oil to the mustard oil by volume is from 5% to 45%.

3. The formulation, as in claim 1, comprising
the proportion of mustard oil and lavender oil in the formulation is from one of (i) 55% mustard oil, 45% Lavender oil, (ii) 60% mustard oil, 40% lavender oil, (iii) 65% mustard oil, 35% lavender oil, (iv) 70% mustard oil, 30% lavender oil, (v) 75% mustard oil, 25% Lavender oil, (vi) 80% mustard oil, 20% lavender oil, (vii) 85% mustard oil, 15% lavender oil, (ix) 90% mustard oil, 10% lavender oil, (x) 95% mustard oil 5% lavender oil.

4. The formulation as in claim 1, comprising:
the formulation is packaged in a container that holds a limited quantity of the formulation, wherein the container has a neck and a dropper to flow drops of the oil for dropping into an ear canal.

5. The formulation as in claim 1, further comprising:
the container is of material for warming the formulation before application, wherein the formulation is warmed up to in the range of 110 to 115 degrees to provide a therapeutic effect as the oil is dropped in the ear canal.

6. The formulation as in claim 5, further comprising:
the formulation is warmed just before use by putting the container in hot water.

7. The formulation as in claim 5, further comprising:
printed instructions for the use of the formulation, including dosage, and advisories, in a package of the formulation.

8. The formulation of claim 1 wherein the proportion of mustard oil to lavender oil is 85% mustard oil and 15% lavender oil.

9. The formulation of claim 1 wherein the proportion of mustard oil to lavender oil is 80% mustard oil and 20% lavender oil.

10. The formulation of claim 1 wherein the proportion of mustard oil to lavender oil is 75% mustard oil and 25% lavender oil.

11. A method for treating middle ear infections, comprising the steps of:
blending a formulation of mustard oil and lavender essential oil;
using a dropper to place drops of the formulation into a human ear canal for treating middle ear infections.

12. The method of claim 11, wherein the step of blending comprises:
blending a proportion in the formulation of lavender oil to the mustard oil wherein the lavender oil from 5% to 45% by volume.

13. The method of claim 11, wherein the step of blending comprises:
blending the proportion of mustard oil and lavender oil in the formulation from one of (i) 55% mustard oil, 45% Lavender oil, (ii) 60% mustard oil, 40% lavender oil, (iii) 65% mustard oil, 35% lavender oil, (iv) 70% mustard oil, 30% lavender oil, (v) 75% mustard oil, 25% Lavender oil, (vi) 80% mustard oil, 20% lavender oil, (vii) 85% mustard oil, 15% lavender oil, (ix) 90% mustard oil, 10% lavender oil, (x) 95% mustard oil 5% lavender oil.

14. The method of claim 11, further comprising the steps of:
packaging the formulation in a container that holds a limited quantity of the formulation, wherein the container has a neck and the dropper to flow drops of the formulation for dropping into the human ear canal.

15. The method of claim 11, further comprising the steps of:
warming the formulation to a range of 110 to 115 degrees to provide a therapeutic effect as the formulation is dropped in the human ear canal.

16. The method as in claim 15, comprising the steps of:
warming the formulation by placing a container in hot water.

17. The method as in claim 15, further comprising the steps of:
providing printed instructions for use of the formulation, including dosage, and advisories, in a package of the formulation.

18. The method of claim 11 wherein the formulation comprises a proportion of mustard oil to lavender oil that is 85% mustard oil and 15% lavender oil.

19. The method of claim 11 wherein the formulation comprises a proportion of mustard oil to lavender oil that is 80% mustard oil and 20% lavender oil.

20. The method of claim 11 wherein the formulation comprises a proportion of mustard oil to lavender oil that is 75% mustard oil and 25% lavender oil.
